(19) United States
(12) Patent Application Publication
(10) Pub. No.: US 2013/0023938 A1
(43) Pub. Date: Jan. 24, 2013

(54) BONE FIXATION WITH OVERLAPPING BONE PLATES

(71) Applicants: Randall J. Huebner, Portland, OR (US); Steven P. Horst, Dayton, OR (US); Matthew J. Ryan, Beaverton, OR (US)

(72) Inventors: Randall J. Huebner, Portland, OR (US); Steven P. Horst, Dayton, OR (US); Matthew J. Ryan, Beaverton, OR (US)

(73) Assignee: ACUMED LLC, Hillsboro, OR (US)

(21) Appl. No.: 13/187,395

(22) Filed: Jul. 20, 2011

(51) Int. Cl. A61B 17/88

(52) U.S. Cl. 606/281

(57) ABSTRACT

System, including methods, apparatus, and kits, for bone fixation using overlapping bone plates each including a plurality of notches arranged along the bone plate to provide interchangeable overlap locations.

Bone plates are low-profile fixation devices, often made of metal, that are mounted on bone with fasteners, to span a fracture (or other bone discontinuity). Many bone plates are at least generally linear, making these plates particularly suitable for placement along the shaft of a long bone.

A fractured pelvis also can be fixed with bone plates. However, because the pelvis is large and shaped irregularly, the fracture pattern can be complex. The pelvis may be fractured multiple times along and across bone at various angles and positions, presenting a substantial fixation challenge to the surgeon. One approach is to attach individual bone plates to nonoverlapping regions on the pelvis, at appropriate orientations, without any mechanical connection between the plates. However, with this approach, it may not be possible to situate all of the plates optimally due to competition for space on bone. Also, stabilization of the pelvis may be inadequate because the plates do not interact directly with each other.

An adaptable and robust bone plate system, with a low profile, is needed for fixation of flat/irregular bones.

### SUMMARY

The present disclosure provides a system, including methods, apparatus, and kits, for bone fixation using overlapping bone plates each including a plurality of notches arranged along the bone plate to provide interchangeable overlap locations.

### BRIEF DESCRIPTION OF THE DRAWINGS

FIG. 1 is a generally anterior, fragmentary view of the left side of a pelvis fixed with a lattice formed of overlapping bone plates, in accordance with aspects of the present disclosure.

FIG. 2 is a plan view of another exemplary lattice that may be formed with copies of one of the bone plates of FIG. 1, taken in the absence of bone, in accordance with aspects of the present disclosure.

FIG. 3 is a sectional view of the lattice of FIG. 2, taken generally along line 3-3 of FIG. 2, with bone screws omitted, in accordance with aspects of the present disclosure.

FIG. 4 is a fragmentary portion of the sectional view of FIG. 3, taken generally at the region indicated at “4” in FIG. 3.

FIG. 5 is another fragmentary portion of the sectional view of FIG. 3, taken generally at the region indicated at “5” in FIG. 3.

FIG. 6 is a fragmentary sectional view of the lattice of FIG. 2, taken generally along line 6-6 of FIG. 2.

FIG. 7 is a side view of an exemplary locking fastener that may be used with the overlapping bone plates disclosed herein, in accordance with aspects of the present disclosure.

FIG. 8 is a side view of an exemplary toggle fastener that may be used with the overlapping bone plates disclosed herein, in accordance with aspects of the present disclosure.

FIG. 9 is a side view of an exemplary lag screw that may be used to attach a pair of overlapping bone plates to each other without extending into bone, in accordance with aspects of the present disclosure.

FIG. 10 is a plan view of an exemplary set of notched bone plates that may be overlapped with one another, in accordance with aspects of the present disclosure.

FIG. 11 is a bottom view of a second embodiment of a notched bone plate for use in an overlapping bone plate system, in accordance with aspects of the present disclosure.

FIG. 12 is a longitudinal sectional view of the notched bone plate of FIG. 11, taken generally along line 12-12 of FIG. 11.

FIG. 13 is a cross-sectional view of the notched bone plate of FIG. 11, taken generally along line 13-13 of FIG. 11.

FIG. 14 is a bottom view of an exemplary overlap configuration that may be produced with two copies of the notched bone plate of FIG. 11, in accordance with aspects of the present disclosure.

FIG. 15 is a plan view of an exemplary overlap configuration that may be produced with two copies of a third embodiment of a notched bone plate, in accordance with aspects of the present disclosure.

FIG. 16 is a plan view of the bottom bone plate copy from the overlap configuration of FIG. 15.

FIG. 17 is a side view of the bottom bone plate copy of FIG. 16.

FIG. 18 is a plan view of a fourth embodiment of a notched bone plate for use in an overlapping bone plate system, in accordance with aspects of the present disclosure.

FIG. 19 is a side, partially sectional view of the notched bone plate of FIG. 18, taken generally along line 19-19 of FIG. 18.

FIG. 20 is a plan view of an exemplary overlap configuration that may be produced with two copies of the notched bone plate of FIG. 18.

FIG. 21 is a plan view of an exemplary lattice that may be produced with copies of a fifth embodiment of a notched bone plate, in accordance with aspects the present disclosure.

FIG. 22 is a plan view of one of the bone plate copies from FIG. 21.

FIG. 23 is a longitudinal sectional view of the bone plate copy of FIG. 22, taken generally along line 23-23 of FIG. 22.

FIG. 24 is a view of a set of exemplary attachments, each of which may be configured to be overlapped with any of the notched bone plates disclosed herein, in accordance with aspects of the present disclosure.

FIG. 25 is a plan view of a sixth embodiment of a notched bone plate, in accordance with aspects of the present disclosure.

FIG. 26 is a side view of the notched bone plate of FIG. 25.

### DETAILED DESCRIPTION

The present disclosure provides a system, including methods, apparatus, and kits, for bone fixation using overlapping bone plates each including a plurality of notches arranged along the bone plate to provide interchangeable overlap locations.

A method of bone fixation is provided. In the method, a first bone plate and a second bone plate may be selected. Each bone plate may define a plurality of notches arranged along the bone plate. For example, each bone plate may define at least three notches arranged along the bone plate. The notches of a bone plate may be formed in only one or in both opposing surfaces (e.g., inner and outer surfaces) of the bone plate. Each notch of the second bone plate may be interchangeably receivable in each notch of the first bone plate to produce a plurality of discrete overlap configurations. The bone plates may be arrangeable obliquely and orthogonally to one another in each overlap configuration. Each overlap configuration may have a collective height that is not substantially greater than a height of one or both of the bone plates individually. Alternatively, or in addition, each overlap configuration may have a collective height that is at least substantially the same as a height of one or both bone plates individually. The bone plates may be disposed on bone in one of the overlap configurations.

Another method of bone fixation is provided. In the method, a first bone plate, a second bone plate, and a third bone plate may be selected. Each bone plate may define a plurality of notches arranged along the bone plate. Each notch of each bone plate may be interchangeably receivable in each notch of each other bone plate to produce a plurality of discrete overlap configurations. The first, second, and third bone plates may be disposed on bone with a notch of the second bone plate and a notch of the third bone plate received in respective notches of the first bone plate.

A system for bone fixation is provided. The system may comprise a first bone plate and a second bone plate each defining a plurality of notches arranged along the bone plate. Each notch of the second bone plate may be interchangeably receivable in each notch of the first bone plate to produce a plurality of discrete overlap configurations. The bone plates may be arrangeable obliquely and orthogonally to one another in each overlap configuration. Each overlap configuration may have a collective height that is at least substantially the same as a height of one or both bone plates individually.

Further aspects of the present disclosure are provided in the following sections: (I) overview of an exemplary fixation system, (II) exemplary notched bone plates, (III) exemplary fasteners for notched bone plates, (IV) exemplary kits with notched bone plates, (V) methods of fixing bones with overlapping bone plates, and (VI) examples.

### I. OVERVIEW OF AN EXEMPLARY FIXATION SYSTEM

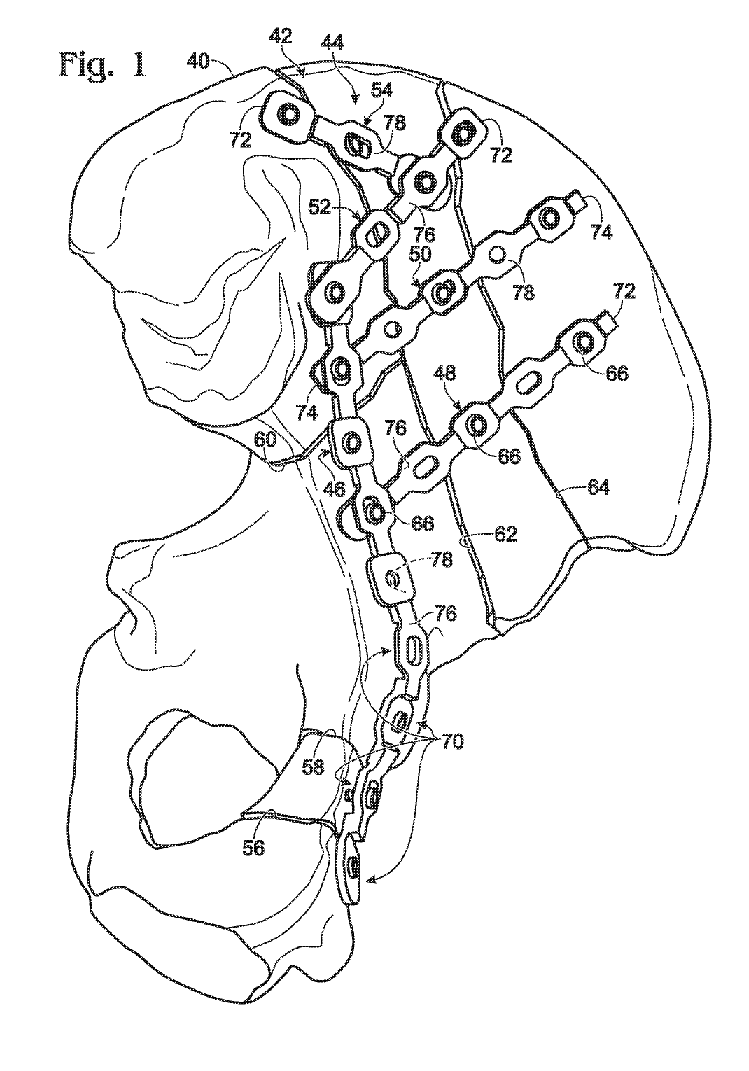

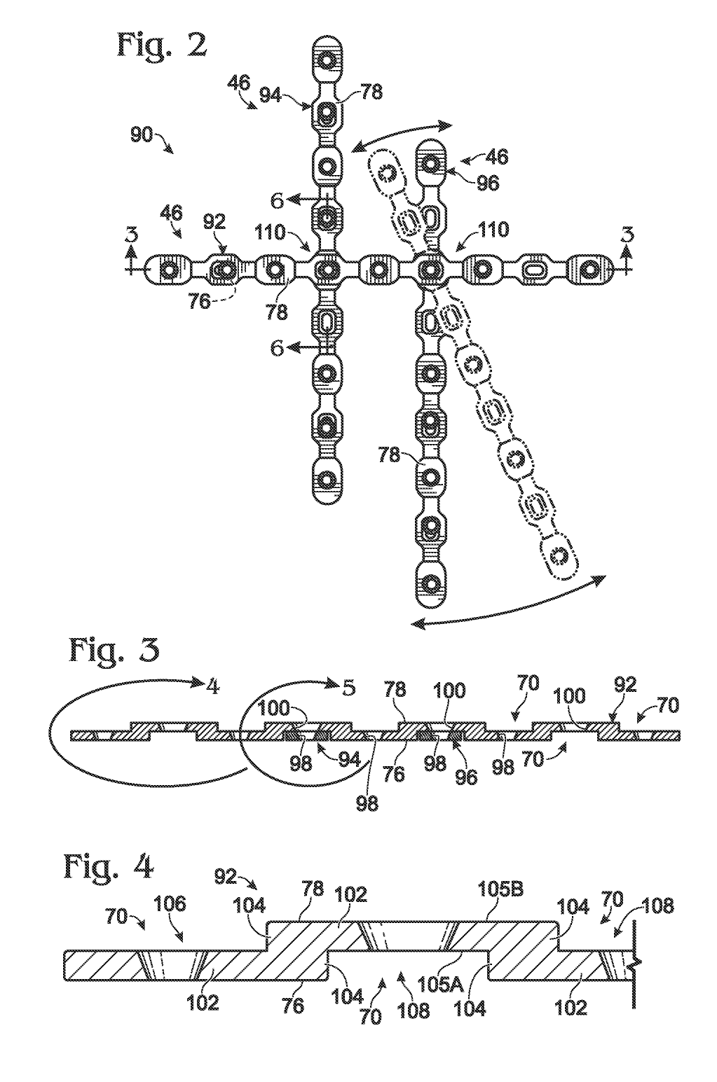

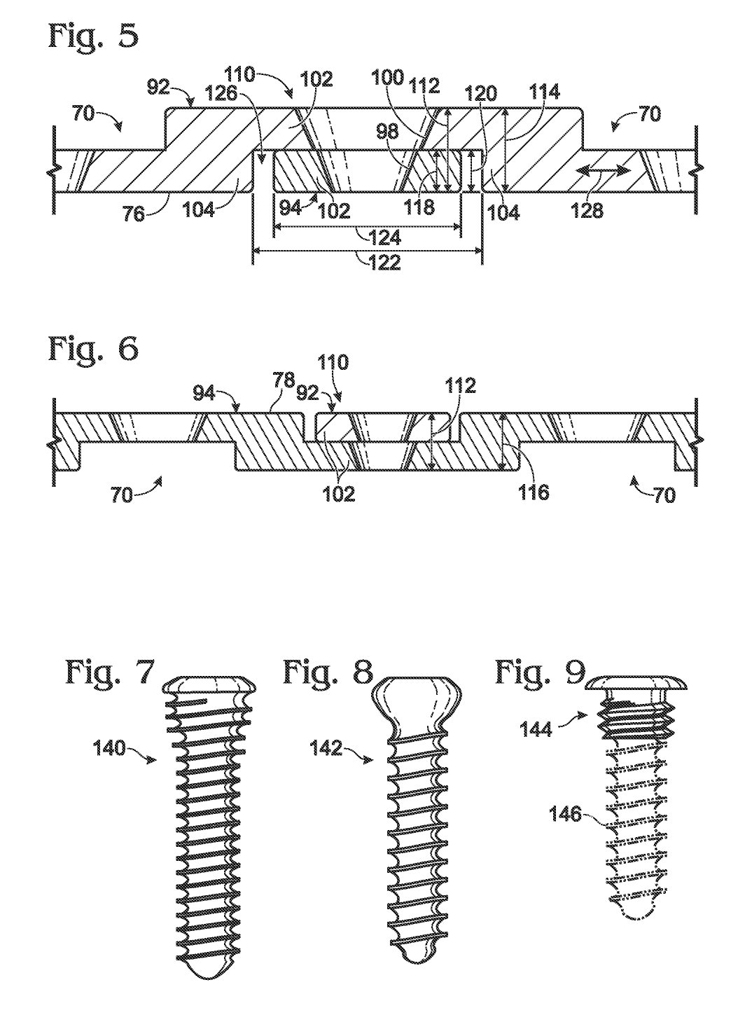

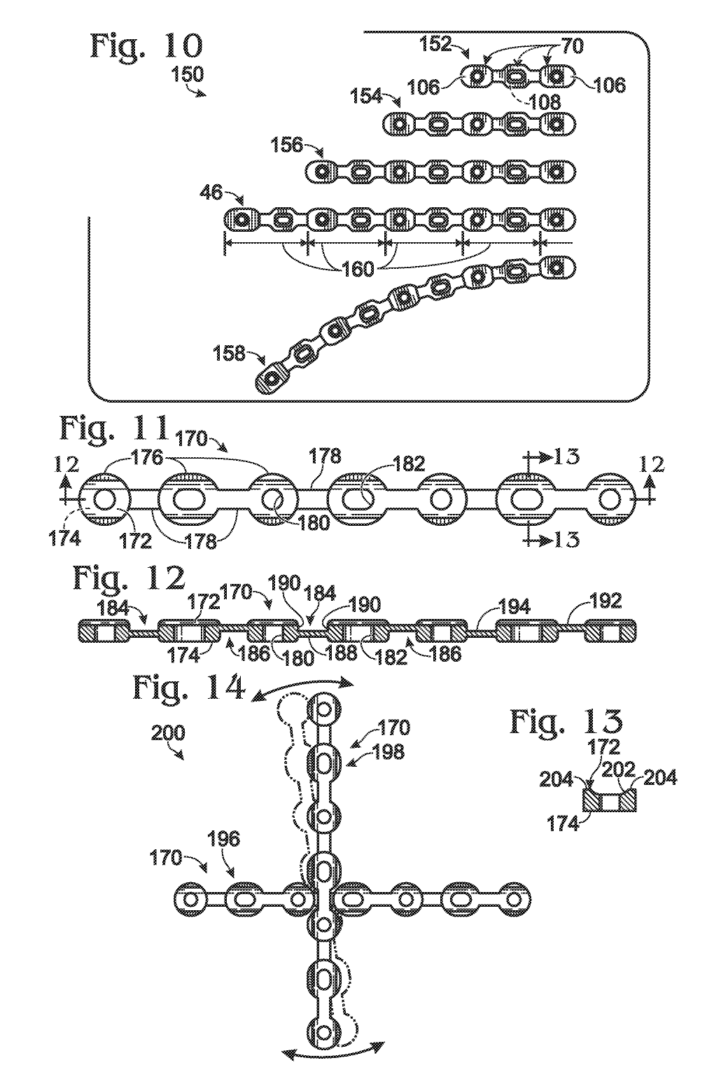

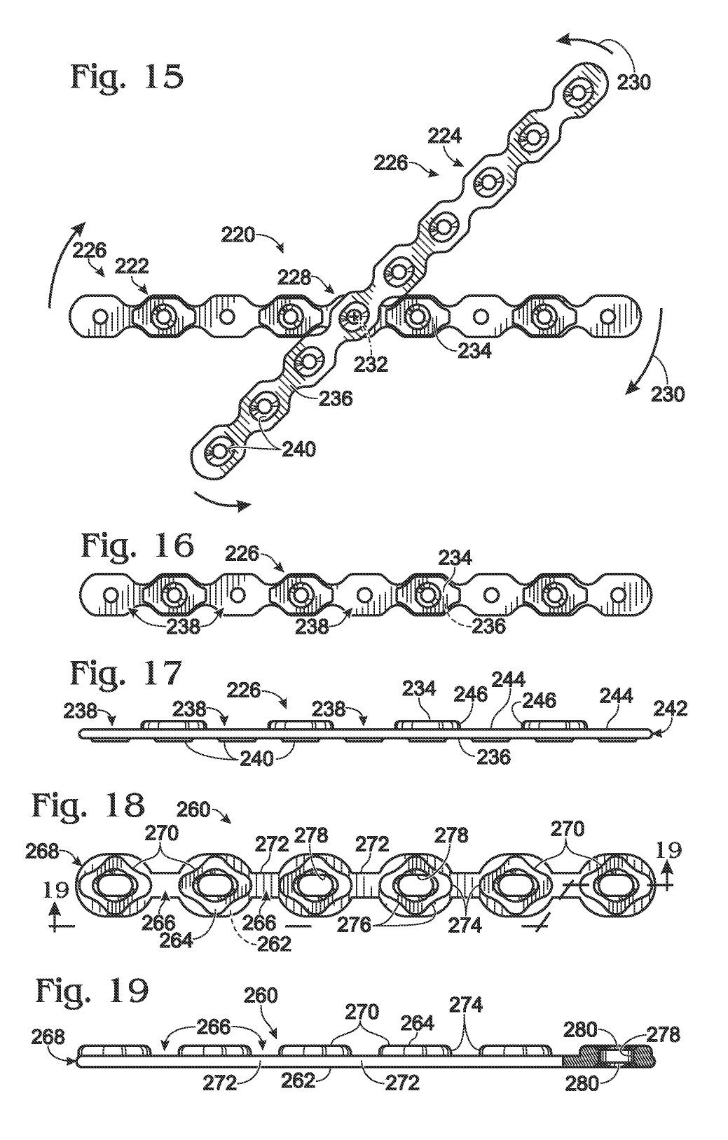

FIG. 1 shows a portion of a multiply-fractured bony pelvis 40, particularly a left hip bone thereof, fixed with a system 42 comprising a lattice 44 composed of overlapping bone plates 46-54. Pelvis 40 has sustained five fractures 56-64 that break the pelvis into the same number of pieces. The bone plates may be attached to bone and/or to one another with fasteners, such as bone screws 66 received in apertures of the bone plates. Each bone plate may overlap one or more other bone plates, with each pair of overlapped bone plates extending crosswise to one another. For example, in the present illustration, bone plate 46 is overlapped by each of bone plates 48-52, and bone plate 52 is also overlapped by bone plate 54.

The lattice may be custom-built as desired by the surgeon according to the needs of the patient. The number of bone plates, the sites of overlap, the relative angular disposition of each pair of bone plates, and the final length of each bone plate may be selected and/or modified to customize the lattice according to the particular bone and/or fracture pattern of the bone and/or the need to reinforce some fractures or regions of the bone more than others. For example, only one bone plate, plate 46, spans fractures 56-60, where stability of the bone may be less problematic, while several bone plates span both of fractures 62 and 64, where stabilization may be more critical. The lattice generally enables the bone plates to function cooperatively as a unit in bone fixation, to achieve better stabilization of complex fixation situations than individual plates working independently. In other embodiments, any of the bone plates disclosed herein may be used to fix any other suitable bone, such as a flat/irregular bone (e.g., a cranium, scapula, sternum, mandible, maxilla, etc.) or a long bone (e.g., a tibia, fibula, femur, humerus, ulna, radius, etc.), among others.

Each bone plate 46-54 includes a series of notches 70 arranged along the plate. The notches provide interchangeable locations of overlap for each bone plate. In other words, each pair of bone plates may be fitted together by placing any notch of one member of the pair into any notch of the other member of the pair. Each pair-wise assembly of notches produces a discrete overlap configuration at a selected overlap location (i.e., a notch) along each plate. The notches may be sized and shaped to minimize or avoid the increase in height that typically occurs when one bone plate is placed over another bone plate. Also, the notches may be sized and shaped to permit assembly of the pair of bone plates at different angular dispositions, such as orthogonally and obliquely to one another in each overlap configuration. Furthermore, assembly of a pair of bone plates at a pair of notches may restrict longitudinal motion of each bone plate in one or both axial directions of the plate, which may interlock the plates.

Lattice 44 may be constructed with a set of bone plates of corresponding structure. For example, the lattice may be built with copies of the same bone plate, such as copies of bone plate 46, with the same number of notches (or an elongated or truncated analog of the plate with a more or fewer notches, respectively). Each copy optionally may be contoured (e.g., bent) intraoperatively to match the contour of a target surface region of pelvis 40 (or other bone) and optionally may be shortened, such as by cutting, to remove part of the bone plate (e.g., to remove one or more notches). In lattice 44, each of bone plates 48, 52, and 54 has been generated by cutting a copy of plate 46 at one position, indicated at 72, and bone plate 50 has been generated by cutting a copy of plate 46 at two positions, indicated twice at 74. In other embodiments, the bone plates may be manufactured with different lengths (e.g., see Section IV).

Bone plate 46 (and modified copies thereof) may be disposed on pelvis 40 interchangeably with either broad surface or face (i.e., the inner/outer surface) disposed adjacent bone. In other words, the bone plate may include opposing surfaces 76, 78 (e.g., see bone plate 46) that are interchangeably arrangeable as an inner surface and an outer surface, or vice versa, by turning over the bone plate. Accordingly, in some embodiments, each opposing broad surface or face of the bone plate may function either as an inner surface or an outer surface, as desired or needed. In lattice 44, bone plates 46, 48, and 52 have surface 76 as the outer surface, and are each inverted relative to bone plates 50 and 54, which have surface 78 as the outer surface.

### II. EXEMPLARY NOTCHED BONE PLATES

FIGS. 2 and 3 show views of another exemplary lattice 90 that may be formed with copies of bone plate 46, which are designated individually here as 92-96 to distinguish the copies. In lattice 90, each of the bone plates is oriented with surface 76 as the inner surface and surface 78 as the outer surface. Each of bone plates 94, 96 is disposed under bone plate 92 in the depicted arrangement.

Each bone plate 92-96 may define one or more circular apertures 98 and/or one or more axially-elongated apertures or slots 100 arranged along the bone plate (see FIG. 3). For example, here, circular apertures 98 and slots 100 alternate along the bone plate. Each aperture may extend from surface 76 to surface 78. The aperture may (or may not) be a locking aperture (e.g., including an internal thread or flange(s) for threaded engagement with an external thread of a fastener). Each aperture may (or may not) overlap a notch, as shown here. Each aperture may (or may not) include a counterbore disposed at one or both opposing ends of the aperture. A counterbore, if present, may be formed in surface 76, surface 78, and/or contiguous with a notch. The counterbore may engage the head of a fastener and/or, in the case of a counterbore of slot 100, may form a ramp along which the head can travel to encourage compression of bone in a direction parallel to the long axis of the plate.

FIG. 4 shows an end region of bone plate 92. One or more of notches 70 may be formed in each surface 76, 78 (or in only one of the opposing surfaces (e.g., see Examples 2 and 3)). In the depicted embodiment, the notches are formed along the plate alternately in surfaces 76 and 78. Each notch may be bounded by a base wall 102 and one or more side walls 104. Base wall 102 may provide opposing surface regions 105A, 105B that respectively form a base surface region of the notch and an opposing-surface region of surface 76 or 78. A notch may be an end notch, indicated at 106, formed at one or both opposing ends of the plate and bounded by only one side wall 104. Also or alternatively, one or more intermediate notches, indicated at 108, may be formed more centrally along the bone plate, with each intermediate notch bounded by a pair of opposing side walls 104 arranged along the plate relative to each other.

FIGS. 5 and 6 show a site of overlap 110 of bone plates 92 and 94 (also see FIG. 2) viewed in respective orthogonal directions. At the site of overlap, a pair of notches are received by one another, with a region of one of the bone plates, namely, base wall 102 of one of the notches (from bone plate 92) disposed over a region of the other bone plate, namely, base wall 102 of the other notch (from bone plate 94). The bone plates assembled (or a lattice formed by three or more of the bone plates) may have a collective height that is less than double the height of one or both (or all) bone plates individually. The collective height may be substantially the same as, and/or not substantially greater than, at least one or both (or all) of the plates individually (e.g., a collective height that is no more than about 25% or 10% greater than the height of one or both (or all) of the bone plates individually). In the depicted embodiment, bone plates 92, 94 have a collective height 112 that is the same as individual heights 114, 116 of bone plates 92 and 94, respectively.

Here, the collective height is not increased because each base wall 102 at the site of overlap is received sufficiently in a notch 70 of the other bone plate. As a result, base wall 102 of bone plate 94 does not project below surface 76 of bone plate 92 (see FIG. 5) and base wall 102 of bone plate 92 does not project above surface 78 of bone plate 94 (see FIG. 6). Each base wall 102 at overlap site 110 may (or may not) be flush (as shown here) or recessed with respect to flanking regions of surface 76 or 78 of the other bone plate. Generally, at a site of overlap, each base wall has a thickness 118 (or other characteristic dimension measured parallel to the plate's height) that is not substantially greater than (e.g., not more than about 20% or 50% greater than) a depth 120 of the notch (also measured parallel to the plate's height) (see FIG. 5). In other words, thickness/dimension 118 of the base wall may be about the same as, or less than, depth 120. In some embodiments, among a set of two or more bone plates, each base wall 102 partially defining a notch may have the same thickness, and/or each notch may have the same depth. Also, each notch of each bone plate may have a depth that is about, or at least about, one-half of the height of the bone plate.

FIG. 2 shows bone plate 96 oriented orthogonally (solid lines) and obliquely (in phantom) relative to bone plate 92. More generally, each overlap configuration of a pair of bone plates may permit the relative angular disposition of the bone plates to be changed (generally, before at least one of bone plates is fully secured to bone) without substantially increasing the collective height of the plates. In other words, one or both notches at a site of overlap can receive the base wall of the other notch at different orientations, and/or permit pivotal movement of the corresponding bone plate after the base wall has been received in a notch. In some cases, the relative angular disposition of a pair of bone plates may be adjusted, optionally continuously, by at least 20, 40, or 60 degrees. For example, a bone plate may be adjustable by at least 10 degrees from orthogonal in either pivotal direction, for a total of at least 20 degrees of adjustment. Adjustment may be achieved by pivotal motion of either or both overlapped plates about an axis extending orthogonally through overlap site 110 (i.e., extending parallel to the axis for measuring the collective height).

FIG. 5 illustrates exemplary dimensional relationships between notch 70 and base wall 102 received in the notch. Each notch may (or may not) have a width 122 (or other characteristic dimension), measured along the bone plate between side walls 104, that is significantly greater than a width 124 of base wall 102 received by the notch (and measured across the plate), to form at least one gap 126. The gap may permit angular adjustment of the overlapped bone plates and some longitudinal and lateral play in plate position before the plates are secured with fasteners. However, contact of the base wall 102 of one plate (e.g., plate 92) with one side wall or opposing side walls 104 of the other plate (e.g., plate 94) at overlap site 110 generally restricts longitudinal motion, indicated by a motion arrow at 128 in FIG. 5 for plate 92. Longitudinal motion may be restricted in one or both longitudinal directions of the plate.

FIG. 5 illustrates overlap site 110 in which slot 100 of plate 92 is disposed over circular aperture 98 of plate 94. In other words, slot 100 is disposed as an outer aperture and circular aperture 98 as an inner aperture. Slot 100 may be utilized to adjust the longitudinal position of plate 92. For example, during installation of the bone plates, bone plate 92 may be pulled longitudinally, from one side of overlap site 110, with a fastener disposed in the slot and the bone plate attached to a different bone fragment on the other side of the overlap site, to compress bone. Alternatively, driving the fastener into the overlapped apertures may drive longitudinal motion of bone plate 92 if slot 100 is configured as a compression slot. In other overlap configurations, a slot may be disposed over another slot, or a circular aperture over another circular aperture or a slot.

Bone plates generally comprise any relatively low-profile (or plate-like) fixation devices configured to be implanted under soft tissue to stabilize at least one bone by attachment to the bone. The fixation devices may be configured to span any suitable bone discontinuity (or discontinuities) so that the fixation devices fix the relative positions of bone fragments disposed on opposing sides of the bone discontinuity (or discontinuities). Alternatively, or in addition, the fixation devices may reinforce a bone lacking a discontinuity.

Suitable discontinuities may occur naturally and/or may result from injury, disease, and/or surgical intervention, among others. Accordingly, exemplary discontinuities for use with the fixation devices disclosed herein may include fractures (breaks in bones), osteotomies (cuts in bones), joints, and/or nonunions (for example, produced by injury, disease, or a birth defect), among others.

The bone plates disclosed herein may be configured for use on any suitable bone, in any suitable vertebrate species, including human, equine, canine, and feline species, among others. Exemplary bones may include bones of the arms (radius, ulna, humerus), legs (femur, tibia, fibula), hands/wrists (e.g., phalanges, metacarpals, and carpals), feet/ankles (e.g., phalanges, metatarsals, and tarsals), vertebrae, scapulas, the pelvis (e.g., hip bones), cranial bones, mandibles, ribs, and/or clavicles, among others. Particular fractures where the bone plates described herein may be suitable include bones with multiple fractures creating a plurality of bone fragments.

The bone plates may be formed of any suitable material(s). The bone plates may be of a sturdy yet malleable construction. Generally, the bone plates should be stiffer and stronger than the section of bone spanned by the plates, yet flexible (e.g., springy) enough not to strain the bone significantly. A bone plate of the present disclosure may be formed of any suitable biocompatible material(s) and/or bioresorbable material(s). Exemplary biocompatible materials that may be suitable for the bone plate include (1) metals/metal alloys (for example, titanium or titanium alloys, alloys with cobalt and chromium (such as cobalt-chrome), stainless steel, etc.); (2) plastics (for example, ultra-high molecular weight polyethylene (UHMWPE), polymethylmethacrylate (PMMA), polytetrafluoroethylene (PTFE), polyetheretherketone (PEEK), and/or PMMA/polyhydroxyethylmethacrylate (PHEMA)); (3) composites (for example, carbon-fiber composites); (4) bioresorbable (bioabsorbable) materials or polymers (for example, polymers of α-hydroxy carboxylic acids (e.g., polylactic acid (such as PLLA, PDLLA, and/or PDLA), polyglycolic acid, lactide/glycolide copolymers, etc.), polydioxanones, etc.; and/or the like.

The plates may be generally elongate, with a length L, a width W, and a thickness T. Here, length L≧width W>thickness T. In use, the long axis of a bone plate may be aligned with the long axis of the corresponding bone, and/or may extend obliquely and/or transversely relative to the bone's long axis. The length and/or width of the bone plates may be varied according to the intended use, for example, to match the plates with a size of bone and/or a particular injury to the bone.

Each plate may define a plurality of apertures. The apertures may have any suitable positions. The apertures may be arrayed generally in a line along a portion of the plate, for example, centered across the width of the plate. Alternatively, the apertures may be arranged nonlinearly, for example, disposed in an arcuate, staggered, or other arrangement.

### III. EXEMPLARY FASTENERS FOR NOTCHED BONE PLATES

FIGS. 7-9 show exemplary externally threaded fasteners or screws 140-144 that may be utilized with any suitable bone plates of the present disclosure. The fasteners may be used in any suitable combination to attach two or more bone plates to one another and/or to bone. Each fastener may be received in only a single bone plate aperture or in a pair of overlapped apertures (e.g., a pair of apertures arranged coaxially), among others. Furthermore, a fastener, such as fastener 140 or 142, may extend into bone from only a single bone plate aperture or from a pair of overlapped apertures. A fastener, such as fastener 140 or 144, may lock by threaded engagement to one or more apertures. For example, fastener 140 may extend into bone and may lock to only one aperture that is not overlapped with another aperture, may lock only to an outer aperture of a pair of overlapped outer and inner apertures, or may lock to an inner aperture and/or an outer aperture of a pair of overlapped apertures. A fastener, such as fastener 142, may function as a toggle fastener that can be placed into a single aperture or a pair of overlapped apertures over a range of angles. In some embodiments, a fastener, such as fastener 144, may be used only where a pair of plates overlap, as a lag screw to lock the pair of plates to one another at an overlap site, without extending into bone, by threaded engagement only with an inner aperture of a pair of overlapping apertures. In this way, fastener 144 may press a pair of plates together as the head of fastener 144 is tightened against the outer aperture. In some embodiments, fastener 144 may include an elongated threaded shaft 146, shown here in phantom outline, that is of sufficient length to extend into bone. In some embodiments, fastener 144 may have a thread that extends farther along the fastener's shaft, to lock to both apertures of a pair of overlapped apertures.

### IV. EXEMPLARY KITS WITH NOTCHED BONE PLATES

The present disclosure provides kits for bone fixation. The kit, which may be described as a system, may include any combination of two or more notched bone plates, at least one attachment (e.g., see Example 4), fasteners for securing the notched bone plates to bone, one or more fasteners for attaching the attachment to a notched bone plate and/or to bone, instruments for use with the bone plates/attachment and/or fasteners (e.g., a cutting tool for shortening the bone plates, a bending tool for contouring the bone plates/attachment intraoperatively, a driver for the fasteners, or the like), and instructions for installation of the bone plates and/or use of the kit's components, among others. In some embodiments, the kit may include multiple copies of only one particular embodiment of a notched bone plate. In other words, all of the bone plates in the kit may have the same shape, size, number of notches, number of apertures, etc. For example, the kit may include only multiple copies of bone plate 46 (see FIG. 2) or any other particular plate embodiment disclosed herein. Alternatively, the kit may include two or more bone plates that are not identical copies of each other, that is, having different shapes, lengths, widths, numbers of notches, numbers of apertures, or any combination thereof, among others. In any event, some or all of the components of each kit may be provided in a sterile condition, such as packaged in a sterile container, and/or may be sterilizable (e.g., autoclavable).

FIG. 10 shows an exemplary set 150 of notched bone plates 46, 152-158 that may be provided by a kit and overlapped with one another in any pair-wise combination to form an assembly (such as a lattice) of two or more bone plates on bone. The kit may include only one copy or two or more copies of any of the bone plates and further may include at least one attachment (e.g., see Example 4). Each of bone plates 152-158 may be an analog of bone plate 46 (and one another), with a plurality of notches 70 (i.e., end notches 106 and intermediate notches 108) arranged along the plate and formed by a repeat structure 160 that creates plate 46 (with about 4% repeats of structure 160). However, bone plates 152-158 may differ from plate 46 in length, curvature (in plan view and/or profile), or both. For example, plates 152-156 have different numbers of repeat structures 160 (about 1%, 2%, and 3%, respectively), while plate 158 is curved.

### V. METHODS OF FIXING BONES WITH OVERLAPPING BONE PLATES

The present disclosure provides methods of fixing bones with notched bone plates. The methods may include any suitable combination of the steps presented below in this Section and elsewhere in the present disclosure, performed in any suitable order and any suitable number of times.

A bone for fixation may be selected. Any suitable bone may be selected having one, two, or more discontinuities, such as a flat/irregular bone (e.g., a hip bone) fractured to create at least two or three or more bone fragments.

A pair of notched bone plates may be selected for fixation of the bone. Each bone plate may be configured to be implanted in the body, over bone and under soft tissue. The pair of bone plates may or may not be copies or analogs of one another, and each may or may not have interchangeable inner and outer surfaces. Each notched bone plate may have any suitable combination of the features described elsewhere herein, such as at least two or at least three notches. In some cases, each notched bone plate may include only one, at least one, or exactly two end notches, and/or at least one or two or more intermediate notches. Each notch of each bone plate may be interchangeably receivable in each notch of the other bone plate to produce a plurality of discrete overlap configurations. In each overlap configuration (i.e., each pair-wise assembly of notches), the bone plates may be arrangeable obliquely and orthogonally to one another. For example, the notches of the bone plates may be configured (e.g., sized and shaped) such that the bone plates are pivotable relative to one another about an axis extending orthogonally through a site of overlap produced by each overlap configuration. Alternatively, the bone plates may be arrangeable relative to one another at only one angular disposition (e.g., orthogonally) in each overlap configuration. Each overlap configuration, and, optionally, the relative angular dispositions of the bone plates permitted by each overlap configuration, may have a collective height that is not substantially greater than a height of one or both bone plates individually, that is, no more than about 25% or 10% greater.

The contour of one or more of the selected bone plates may be modified. For example, at least one of the bone plates may be bent intraoperatively. Bending the bone plate may change its shape in profile.

The bone plates may be disposed on the bone in one of the overlap configurations. In the one overlap configuration, one of the bone plates is disposed over the other bone plate (as determined at the site of overlap). In some embodiments, a third notched bone plate may be selected and disposed on the bone. The third bone plate may be overlapped with, and arranged crosswise to, one or both of the other two bone plates. In any event, the angular disposition of the overlapped bone plates may be adjusted after the bone plates are disposed on the bone, and, optionally, after a fastener has been placed into a pair of overlapped apertures at an overlap site formed by the overlapped bone plates.

Each bone plate of the pair may be secured to the bone in the one overlap configuration. Securing the bone plates may be performed by placing one or more fasteners through at least one or two or more openings of each bone plate and into the bone. A fastener may or may not extend into an aperture of each bone plate at the site of overlap. Generally, each bone plate may be secured to a distinct bone fragment on each opposing side of a discontinuity, such as a fracture, in the bone.

One or more of the bone plates selected may be reduced in length (shortened). For example, a bone plate may be cut or broken to remove part of the bone plate, such as at least one notch. The bone plate may be shortened before or after the bone plate is disposed on the bone, disposed in an overlap configuration on bone, and/or secured to the bone.

In some embodiments, an attachment (e.g., see Example 4) may be selected. The attachment may include a notch and may be overlapped with at least one of the bone plates with the notch of the attachment received in a notch of a bone plate. A fastener may be placed through a pair of overlapping apertures defined collectively by the attachment and the bone plate, to attach the attachment to the bone plate.

### VI. EXAMPLES

The following examples describe selected aspects and embodiments of the present disclosure, including methods and apparatus for bone fixation with overlapping bone plates. These examples and the various features and aspects thereof are included for illustration and are not intended to define or limit the entire scope of the present disclosure. Furthermore, any of the aspects and features disclosed in the following examples and elsewhere in the present disclosure (e.g., in relation to FIGS. 1-10) may be associated with one another in any suitable combination.

### Example 1

### Notched Bone Plate with Predefined Inner and Outer Surfaces

This example describes an exemplary notched bone plate 170 with predefined inner and outer surfaces 172, 174, respectively; see FIGS. 11-14. In each of FIGS. 11-14, inner surface 172, which is configured to face bone, is illustrated facing upward or outward. In other embodiments, bone plate 170 may be configured with opposing surfaces that are interchangeably positionable as inner and outer surfaces.

Bone plate 170 forms a plurality of anchor members 176 arranged along the plate and connected by interposed spanning members or neck regions 178. Each anchor member may define an aperture, namely, a circular aperture 180 or a slot 182, which can receive a fastener, such as a bone screw, that attaches the anchor member to bone. Each spanning member 178 is interposed between an adjacent pair of anchor members 176 and connects the adjacent pair to each other. The spanning member may be narrower and/or thinner than the anchor members, to form notches, which may enable cutting/breaking the bone plate at a spanning member to shorten the plate.

FIG. 12 shows a sectional view of bone plate 170. The plate includes a plurality of inner notches 184 and outer notches 186 arranged along the bone plate and alternately formed in inner surface 172 (inner notches 184) and outer surface 174 (outer notches 186). Each notch is defined by an adjacent pair of anchor members and the spanning member disposed between the pair. In particular, a spanning member 178 (see FIG. 11) forms a base wall 188 of the notch, and the adjacent pair of anchor members form opposing side walls 190 of the notch. Spanning members 178 are formed at different heights, such as alternating between lower and higher positions, as shown in FIG. 12, to produce lower walls 192 and higher walls 194. In other embodiments, the spanning members all may have the same height, such that all of the notches are formed in the same surface of the bone plate (e.g., see Example 2).

FIG. 14 shows a bottom view of two copies 196, 198 of bone plate 170 arranged in an overlap assembly 200. An inner notch 184 of bone plate copy 196 has been placed into an outer notch 186 of bone plate copy 198, such that a higher wall 194 of copy 196 is disposed over a lower wall of copy 198 (also see FIG. 12). The relative angular disposition of copies 196, 198 is adjustable as indicated by double-headed motion arrows and an obliquely positioned copy 198 in phantom outline. The height of overlap assembly 200 is the same as the height of bone plate 170.

FIGS. 11 and 13 illustrate aspects of inner surface 172 and outer surface 174. The inner surface includes a concave surface region 202 (i.e., concave transversely) flanked laterally by flats 204. Flats 204 contact bone, and surface region 202 is elevated from bone, which may minimize the surface area of periosteum engaged, and potentially damaged, by the bone plate. In other embodiments, concave surface region 202 and flats 204 may be present on both opposing surfaces of the plate, to allow the bone plate to be used equivalently when turned over.

### Example 2

### Bone Plates with Uni-directional Notches

This example describes exemplary notched bone plates with overlappable notches formed in only one surface of each bone plate; see FIGS. 15-20.

FIG. 15 shows an exemplary overlap configuration 220 that may be produced with two copies 222, 224 of a third embodiment of a notched bone plate 226. Bone plate copy 224 extends over bone plate copy 222 in configuration 220, to form an overlap site 228 where a region of copy 222 is disposed directly under a region of copy 224. The overlap site is produced by disposing a notch of one of the bone plate copies in a notch of the other bone plate copy. The angular disposition of the bone plate copies relative to each other is adjustable in the overlap configuration, as indicated by motion arrows 230. Either or both bone plate copies may be moved pivotally, about an axis 232 extending orthogonally through overlap site 228, such as coaxial with overlapped apertures at the overlap site. In some cases, a fastener may be placed into the overlapped apertures, and optionally into bone, before one or both bone plates are fully secured to bone. The fastener then may provide a shaft about which one or both plates can be pivoted.

FIGS. 16 and 17 show respective plan and side views of bone plate 226. The bone plate includes opposing surfaces, namely notched surface 234 and footed surface 236. Notched surface 234 has a plurality of notches 238 formed therein. Footed surface 236 has a plurality of feet 240 formed thereon and projecting in a direction generally opposite to the notches. Respective notched surfaces 234 of the two bone plate copies face in opposite directions in configuration 220 relative to one another, as do respective footed surfaces 236 (see FIG. 15). In any event, feet 240 of bone plate 226 can contact bone, thereby elevating the majority of footed surface 236 above bone, to reduce the surface area of periosteum contacted and potentially injured.

FIG. 17 illustrates how bone plate 226 may be structured. The bone plate can include a bar or baseplate portion 242 providing a recessed portion of footed surface 236 and recessed regions 244 of notched surface 234. Baseplate portion 242 may be planar, with a linear profile. Feet 240 project with respect to the recessed portion of footed surface 236 and islands 246 with respect to recessed surface regions 244, with the feet and islands projecting in opposite directions and at regular positions along their respective surfaces. In any event, the bone plate varies in thickness along it length, with relatively thinner regions (and notches) formed between islands and interspersed with relatively thicker regions created by the islands. The overlap configuration of FIG. 15 may (or may not) produce a small, insubstantial increase in height over the height of either bone plate copy alone, due to a small height increase caused by outwardly-facing feet 240 of one of the bone plate copies projecting above islands 246 of the other bone plate copy.

FIGS. 18 and 19 show respective plan and side views of a fourth embodiment of a notched bone plate 260 for use in an overlapping bone plate system. Plate 260 includes a flat surface 262 that opposes a notched surface 264 forming a plurality of notches 266. The bone plate includes a planar baseplate portion 268 and a plurality of islands 270 projecting from the baseplate portion at uniformly-spaced positions along the baseplate portion. The baseplate portion forms spanning members 272 that connect the islands and provide a base wall of each notch. The islands form opposing side walls 274 of each notch that taper toward the notch. Each island may define a plurality of indentations 276 that are configured to engage a flange of the baseplate portion 268 of another notched plate.

Each island 270 defines a slot 278. The slot may have counterbore structure 280 formed in each opposing surface 262, 264 of the plate (see FIG. 19).

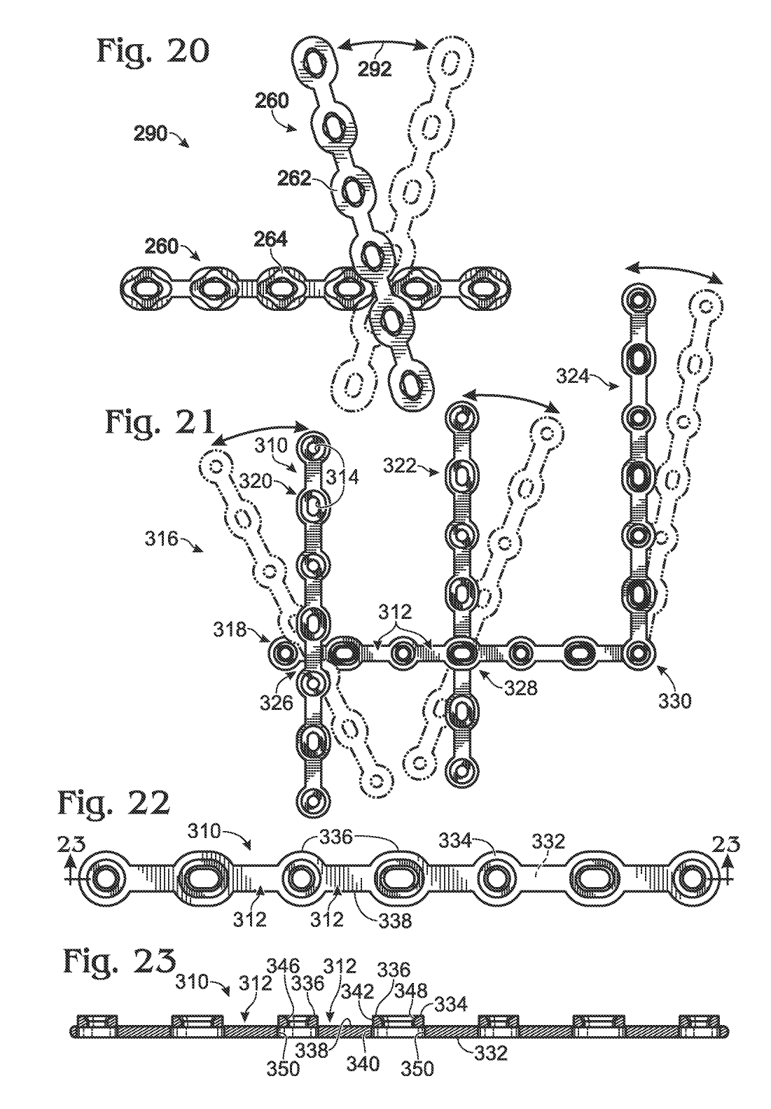

FIG. 20 shows an exemplary overlap configuration 290 that may be produced with two copies of bone plate 260. Flat surface 262 acts as the outer surface for one of the bone plate copies and notched surface 264 for the other copy. In other words, one plate is inverted relative to the other. The bone plates copies are pivotable with respective to each other, indicated at 292 for one of the copies, before they are secured to bone, without increasing the height of the bone plate assembly.

### Example 3

### Notched Bone Plate with Distinct Overlap Modes

This example describes an exemplary notched bone plate 310 configured to be overlapped at one or more locations defined by at least one notch 312 and/or at least one aperture 314; see FIGS. 21-23.

FIG. 21 shows an exemplary lattice 316 that may be produced with copies 318-324 of notched bone plate 310 overlapping one another at overlap sites 326-330. Site 326 produces an overlap configuration of plate copies 318 and 320 in which notches 312 of the copies are mutually received by one another, without substantially increasing the height of either bone plate (e.g., see Examples 1 and 2). Copies 318 and 320 are inverted relative to each other. In contrast, overlap sites 328 and 330 position plate copy 318 respectively over and under plate copies 322 and 324 at coaxially aligned apertures of the respective plate copies, which produces a substantial increase (about 50%) in the collective height of the overlapped bone plates.

FIGS. 22 and 23 show respective plan and sectional views of bone plate 310. The plate includes a flat surface 332 and a notched surface 334 that opposes the flat surface. The plate also includes a plurality of anchor members 336 connected to one another by spanning members 338. Each anchor member includes a base portion 340 and a projecting portion 342. The projecting portion forms a circular or oblong button. The button of each anchor member defines a first region of aperture 314 (see FIG. 21), namely, a circular opening 346 or an elongate opening 348 for receiving a fastener (see FIG. 23). The base portion of each anchor member defines a socket 350 arranged coaxially with opening 346 or 348 and sized to receive a circular button provided by another copy of the plate. In particular, a socket 350 defined under an opening 346 or 348 can receive a circular button formed around a circular opening.

### Example 4

### Attachments for Notched Bone Plates

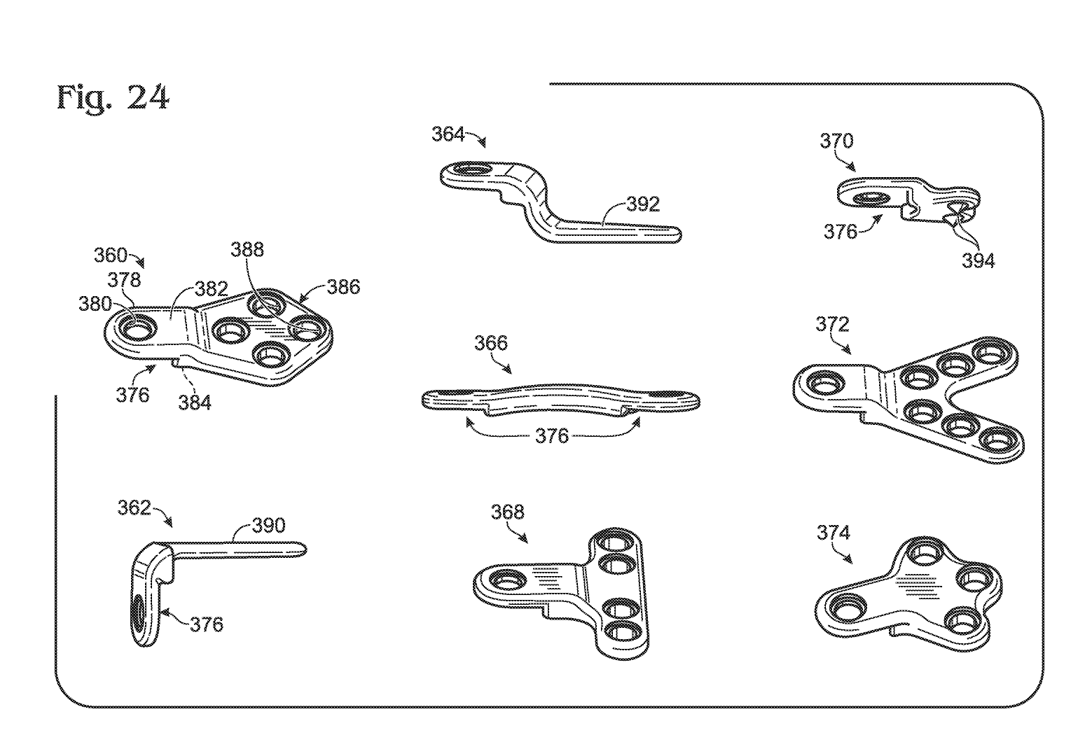

This example describes exemplary attachments 360-374 for overlap with notched bone plates; see FIG. 24.

Each attachment 360-374 may be overlapped with any of the notched bone plates disclosed herein, particularly bone plates with an aperture extending through the plate from each notch (e.g., bone plates 46 and 226; see FIGS. 2, 3, 10, and 15). A bone plate overlapped with an attachment may have a collective height that is at least substantially the same as the height of the bone plate alone. The attachment may include an end notch 376, optionally formed as an undercut (or an overcut), at an end of the attachment, to create a tab or flange 378 (see attachment 360). The end notch may be contiguous with at least one aperture 380 that is defined by the tab and that extends between inner and outer surfaces 382, 384 of the attachment. The tab may be placed into a notch of a bone plate, with aperture 380 aligned coaxially with an aperture defined by a base wall of the bone plate. The attachment may be placed under or over a notched bone plate. A fastener (e.g., see FIGS. 7-9) may be placed through the aligned apertures, to secure the attachment (360-374) to the notched bone plate, which may or may not be overlapped with one or more other notched bone plates and/or attachments.

Each attachment may have any suitable head portion 386 extending from tab 378 and configured for attachment to and/or insertion into bone. The head portion may define one or more apertures 388 (e.g., attachments 360, 366, 368, 372, 374) for receiving fasteners. For example, the head portion may form a quadrilateral (360), a T-shape (368), a Y-shape (372), or a three-lobed extension (374), among others. Alternatively, or in addition, the head portion may provide a blade 390, spike 392, or prong(s) 394 for engagement with and/or placement into bone. Attachment 366 may serve as a bridge that extends between a pair of notched bone plates and/or between a notched bone plate and another attachment.

### Example 5

### Notched Bone Plate with Sloped Side Walls

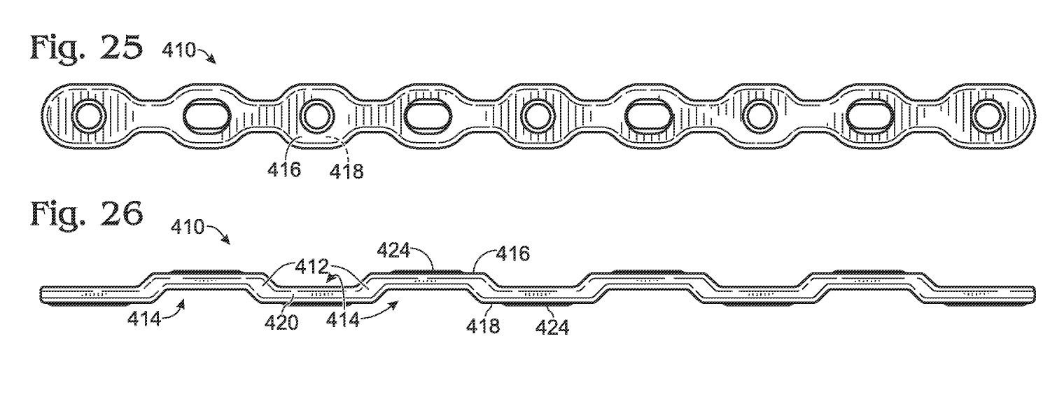

This example describes an exemplary notched bone plate 410 having sloped side walls 412 flanking notches 414; see FIGS. 25 and 26.

Bone plate 410 has features present in bone plate 46 (e.g., see FIGS. 1-6). For example, plate 410 has notches 414 formed alternately in opposing surfaces 416, 418 along the plate. Accordingly, a first copy of the bone plate can be disposed interchangeably under and over a second copy of the plate without turning either copy over.

Bone plate 410 is distinct from bone plate 46. Each notch is bounded by a base wall 420 and side walls 412 oriented obliquely to the base wall. In particular, the side walls extend obliquely away from the base wall to form a notch that is flared in cross section (e.g., as visible in FIG. 26). Feet 424 are formed on each opposing surface 416, 418, which may elevate the body of the plate above bone whether surface 416 or 418 is facing bone.

### Example 6

### Selected Embodiments

This example describes selected embodiments of the present disclosure, presented as a series of numbered paragraphs.

1. A method of bone fixation, comprising: (A) selecting a first bone plate and a second bone plate each defining a plurality of notches arranged along the bone plate, wherein each notch of the second bone plate is interchangeably receivable in each notch of the first bone plate to produce a plurality of discrete overlap configurations, wherein the bone plates are arrangeable obliquely and orthogonally to one another in each overlap configuration, and wherein, optionally, each overlap configuration has a collective height that is not substantially greater than a height of one or both bone plates individually; and (B) disposing the bone plates on bone in one of the overlap configurations.

2. The method of paragraph 1, wherein longitudinal motion of each bone plate in one or both longitudinal directions of such bone plate is limited by the other bone plate in each overlap configuration.

3. The method of paragraph 1 or 2, wherein the bone plates are pivotable relative to one another in each overlap configuration without substantially increasing the collective height.

4. The method any of paragraphs 1 to 3, wherein the second bone plate is continuously pivotable with respect to the first bone plate in each overlap configuration through a total angle of at least about 20 degrees.

5. The method of any of paragraphs 1 to 4, wherein each bone plate includes three or more notches.

6. The method of any of paragraphs 1 to 5, wherein each bone plate includes a notch formed at each end of such bone plate.

7. The method of any of paragraphs 1 to 6, wherein the overlap configurations are first overlap configurations, further comprising (i) a step of selecting a third bone plate including a plurality of notches each receivable interchangeably in each notch of the first bone plate and in each notch of the second bone plate to define a plurality of discrete second overlap configurations, and (ii) a step of disposing the third bone plate on bone and overlapped with the first bone plate or the second bone plate, or both, in one or more of the second overlap configurations.

8. The method of any of paragraphs 1 to 7, further comprising a step of placing a same fastener in an aperture of each bone plate.

9. The method of any of paragraphs 1 to 8, wherein an aperture extends through a bone plate from each notch.

10. The method of any of paragraphs 1 to 9, wherein each bone plate is narrower at each notch and wider between notches.

11. The method of any of paragraphs 1 to 7 and 10, wherein no aperture extends through either the first bone plate or the second bone plate from one of the notches.

12. The method of any of paragraphs 1 to 11, wherein at least one of the bone plates includes opposing first and second surfaces, and wherein at least one of the notches is formed in the first surface and at least one of the notches is formed in the second surface.

13. The method of paragraph 12, wherein the notches are formed along the plate alternately in the first and second surfaces.

14. The method of any of paragraphs 1 to 13, further comprising a step of shortening at least one of the bone plates to remove one or more notches.

15. The method of paragraph 14, wherein the step of shortening includes a step of cutting the at least one bone plate and is performed after the step of selecting and before and/or after the step of disposing.

16. The method of any of paragraphs 1 to 15, further comprising a step of bending one or both bone plates before and/or after the step of disposing.

17. The method of any of paragraphs 1 to 16, wherein each of the first and second bone plates has a same structure repeated contiguously two or more times along each plate, and wherein each occurrence of the same structure provides one or more of the notches.

18. The method of any of paragraphs 1 to 17, wherein the step of selecting selects first and second bone plates that are copies of each other.

19. The method of any of paragraphs 1 to 18, wherein notches of the first bone plate have a same size, shape, and spacing as notches of the second bone plate.

20. The method of any of paragraphs 1 to 19, wherein each bone plate is configured to be interchangeably disposed under and over the other bone plate.

21. The method of paragraph 20, wherein each bone plate is configured to be interchangeably disposed under and over the other bone plate without turning over either bone plate.

22. The method of any of paragraphs 1 to 6 and 8 to 21, further comprising a step of disposing a third bone plate on the bone and overlapped with and arranged crosswise to the first bone plate, the second bone plate, or both the first and second bone plates.

23. The method of any of paragraphs 1 to 22, wherein the bone is included in a pelvis.

24. The method of any of paragraphs 1 to 23, further comprising a step of fixing the one overlap configuration on the bone with one or more fasteners.

25. A method of bone fixation, comprising: (A) selecting a first bone plate and a second bone plate each defining three or more notches arranged along the bone plate, wherein each notch of the second bone plate is interchangeably receivable in each notch of the first bone plate to produce a plurality of discrete overlap configurations, and wherein the bone plates are arrangeable obliquely and orthogonally to one another in each overlap configuration; and (B) disposing the bone plates on bone in one of the overlap configurations.

26. A method of bone fixation, comprising: (A) selecting a first bone plate and a second bone plate each defining three or more notches arranged along the bone plate, wherein each notch of the second bone plate is interchangeably receivable in each notch of the first bone plate to produce a plurality of discrete overlap configurations, and wherein each overlap configuration has a collective height that is at least substantially the same as a height of one or both bone plates individually; and (B) disposing the bone plates on bone in one of the overlap configurations.

27. A method of bone fixation, comprising: (A) selecting a first bone plate, a second bone plate, and a third bone plate each defining a plurality of notches arranged along the bone plate, wherein each notch of each bone plate is interchangeably receivable in each notch of each other bone plate to produce a plurality of discrete overlap configurations; and (B) disposing the first, second, and third bone plates on bone with a notch of the second bone plate and a notch of the third bone plate received in respective notches of the first bone plate.

28. A system for bone fixation, comprising a first bone plate and a second bone plate each defining a plurality of notches arranged along the bone plate, each notch of the second bone plate being interchangeably receivable in each notch of the first bone plate to produce a plurality of discrete overlap configurations, the bone plates being arrangeable obliquely and orthogonally to one another in each overlap configuration, and each overlap configuration having a collective height that is at least substantially the same as a height of one or both bone plates individually.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure. Further, ordinal indicators, such as first, second, or third, for identified elements are used to distinguish between the elements, and do not indicate a particular position or order of such elements, unless otherwise specifically stated.

We claim:

1. A method of bone fixation, comprising:
selecting a first bone plate and a second bone plate each defining a plurality of notches arranged along the bone plate, wherein each notch of the second bone plate is interchangeably receivable in each notch of the first bone plate to produce a plurality of discrete overlap configurations, and wherein the bone plates are arrangeable obliquely and orthogonally to one another in each overlap configuration; and
disposing the bone plates on bone in one of the overlap configurations.

2. The method of claim 1, wherein each overlap configuration has a collective height that is not substantially greater than a height of one or both bone plates individually.

3. The method of claim 2, wherein the bone plates are pivotable relative to one another in each overlap configuration without substantially changing the collective height.

4. The method of claim 1, wherein each bone plate includes three or more notches.

5. The method of claim 1, wherein the overlap configurations are first overlap configurations, further comprising (a) a step of selecting a third bone plate including a plurality of notches each receivable interchangeably in each notch of the first bone plate and in each notch of the second bone plate to define a plurality of discrete second overlap configurations, and (b) a step of disposing the third bone plate on bone and overlapped with the first bone plate or the second bone plate, or both, in one or more of the second overlap configurations.

6. The method of claim 1, wherein longitudinal motion of each bone plate in one or both longitudinal directions of such bone plate is limited by the other bone plate in each overlap configuration.

7. The method of claim 1, wherein at least one of the bone plates includes opposing first and second surfaces, and wherein at least one of the notches is formed in the first surface and at least one of the notches is formed in the second surface.

8. The method of claim 1, further comprising a step of shortening at least one of the bone plates to remove one or more notches, wherein the step of shortening is performed before and/or after the step of disposing.

9. The method of claim 1, wherein notches of the first bone plate have a same size, shape, and spacing as notches of the second bone plate.

10. The method of claim 1, wherein each bone plate is configured to be interchangeably disposed under and over the other bone plate.

11. The method of claim 10, wherein each bone plate is configured to be interchangeably disposed under and over the other bone plate without turning over either bone plate.

12. The method of claim 1, wherein the bone is included in a pelvis.

13. The method of claim 1, further comprising a step of fixing the one overlap configuration on the bone with one or more fasteners.

14. A method of bone fixation, comprising:
selecting a first bone plate, a second bone plate, and a third bone plate each defining a plurality of notches arranged along the bone plate, wherein each notch of each bone plate is interchangeably receivable in each notch of each other bone plate to produce a plurality of discrete overlap configurations; and
disposing the first, second, and third bone plates on bone with a notch of the second bone plate and a notch of the third bone plate received in respective notches of the first bone plate.

15. The method of claim 14, wherein each overlap configuration has a collective height that is not substantially greater than a height of at least one of the bone plates individually.

16. The method of claim 15, wherein a pair of the bone plates are pivotable relative to one another in each overlap configuration without substantially changing the collective height.

17. The method of claim 14, wherein longitudinal motion of each bone plate in one or both longitudinal directions of such bone plate is limited by the other bone plate in each overlap configuration.

18. The method of claim 14, wherein at least one of the bone plates includes opposing first and second surfaces, and wherein at least one of the notches is formed in the first surface and at least one of the notches is formed in the second surface.

19. The method of claim 14, wherein each bone plate is configured to be interchangeably disposed under and over each other bone plate without turning over a bone plate.

20. A system for bone fixation, comprising:
a first bone plate and a second bone plate each defining a plurality of notches arranged along the bone plate, each notch of the second bone plate being interchangeably receivable in each notch of the first bone plate to produce a plurality of discrete overlap configurations, the bone plates being arrangeable obliquely and orthogonally to one another in each overlap configuration, and each overlap configuration having a collective height that is at least substantially the same as a height of one or both bone plates individually.
